(19) United States
(12) Patent Application Publication
(10) Pub. No.: US 2024/0245892 A1
(43) Pub. Date: Jul. 25, 2024

(54) Non-Invasive and Passive Transdermal Drug Delivery Patch For Parkinson's Disease

(71) Applicant: Georgetown University, Washington, DC (US)

(72) Inventors: Charbel Moussa, Germantown, MD (US); Makarand Paranjape, Ashton, MD (US)

(73) Assignee: Georgetown University, Washington, DC (US)

(21) Appl. No.: 18/293,459

(22) PCT Filed: Oct. 24, 2022
(86) PCT No.: PCT/US22/47589

§ 371 Date: Jan. 30, 2024

Related U.S. Application Data
(60) Provisional application No. 63/303,099, filed on Jan. 26, 2022.
(60) Provisional application No. 63/297,086, filed on Jan. 6, 2022.
(60) Provisional application No. 63/270,690, filed on Oct. 22, 2021.

(51) Int. Cl. A61M 37/00; A61K 31/197

(52) U.S. Cl. CPC A61M 37/00; A61K 31/197; A61M 2037/0007

(57) ABSTRACT

A flexible drug delivery patch is described for non-invasively delivering macromolecular drugs directly to the circulatory system of a user. The patch includes multiple sealed reservoirs formed therein, the sealed reservoirs containing the macromolecular drugs which are entrapped within one of a dissolvable polymer matrix using one of nanoparticles or nanofibers or a thermo-responsive hydrogel. The macromolecular drugs being released from the sealed reservoirs and the entrapping material by activating one or more electrically addressable microheating units.

### CROSS REFERENCE TO RELATED APPLICATIONS

The present application claims the benefit of priority to U.S. Provisional Patent Application Nos. 63/270,690, 63/297,086 and 63/303,099, commonly titled “NON-INVASIVE AND PASSIVE TRANSDERMAL DRUG DELIVERY PATCH FOR PARKINSON'S DISEASE” which were filed on Oct. 22, 2021, Jan. 6, 2022 and Jan. 26, 2022, respectively, and which are incorporated herein by reference in their entirety.

### BACKGROUND

### Field of the Embodiments

The field of the embodiments is, generally, the field of drug delivery, and specifically, the field of skin patch drug delivery systems and methods.

### Description of Related Art

The concept of delivering medications and other health-related substances, e.g., insulin, hormones, nicotine, through the skin, has been of particular interest to the medical community for decades. Numerous transdermal drug delivery devices, utilizing varied approaches, are described in the relevant literature, with a few of such devices reaching the market. But there is currently no one-size-fits-all device. The prior art device designs attempt to balance many factors, such as, patient comfort, delivery efficiency, device size, delivery requirements (e.g., continuous, on-demand, feedback-responsive), and the like. Further, as new medications are developed and disease management and treatment protocols and patient quality of life improve with, e.g., continuous treatment, methods are sought for administering these medications efficiently and with manageable side effects.

For example, while research has continued in an effort to better manage the symptoms of Parkinson's disease (e.g., motor dysfunction), the current gold standard is an orally administered formulation that was first developed over 50 years ago, L-3,4-dihydroxyphenylalanine (L-DOPA), or Levodopa. But as is the case with many oral medications, orally administered L-DOPA is not without side effects. Further, in order to effectively alleviate the symptoms, L-DOPA medications must be maintained at near constant levels in the blood. This is difficult to achieve given that oral medications are prone to problems associated with bioavailability and first-pass effects. Prior art devices which have been developed in an effort to meet these challenges include, e.g., surgical installation of a small, portable infusion pump to deliver a gel-based L-DOPA enteral suspension directly into the small intestine. But this is an invasive procedure. And while a drug delivery patch has been described in the prior art for delivery of an alternative drug to L-DOPA i.e., Rotigotine, Rotigotine is an inferior drug to L-DOPA for treating symptoms of Parkinson's. As early as 2010, the prior art identified a need for a skin patch that could deliver L-DOPA. See, A. Abbott, “Levodopa: The Story So Far,” Nature, 466, p. S6, 2010. But the barrier to therapeutic transdermal delivery of L-DOPA is L-DOPA's lack of passive permeability with respect to the skin. L-DOPA cannot be readily modified for passive transdermal delivery like, for example, nicotine's chemical alteration for use in smoking cessation patches.

The skin's physiological barrier effectively limits passive transdermal delivery to low molecular weight and lipophilic drugs. The delivery of macromolecular medication remains challenging since their size restricts easy transport across the stratum corneum (SC), the dry, brick-and-mortar style cell arrangement of the topmost layer of the epidermis. The low permeability of the SC to macromolecules, hydrophilic and water-soluble drugs presents a major limitation of TDDS that must be overcome to allow a broader spectrum of drug administration through the skin. In order for any drug to be absorbed by passive diffusion through the skin, its molecular weight should be less than 500 Daltons (Da) and should be lipophilic. Passive diffusion usually takes place when a drug travels through the skin via an intercellular route, i.e., along a tortuous pathway that winds around stratum corneum (SC) corneocyte cells.

Alternatively, and to a lesser degree, drugs can travel along a transappendageal pathway, which relies on transport through pores of hair follicles or eccrine sweat glands. Although L-DOPA has a molecular weight just under 200 Da, it is very hydrophilic. As such, alternative transdermal delivery systems have been described and attempted which require either invasive transdermal procedures (e.g., skin permeators such as microneedles, laser ablation, iontophoresis, and pressurized injections) which are cumbersome and cause injury to the skin and/or require extremely complex drug modification including encapsulation within “stealth-carriers.” Such a transdermal delivery typically makes use of an intracellular route, where the drug must diffuse through multiple levels of corneocytes and intermediate lipid layers to eventually reach the circulatory system. The drugs must therefore be modified using chemical enhancers to allow for their permeation through the different hydrophobic/hydrophilic layers of the SC.

Accordingly, there remains an identified need in the art for a skin patch-type non-invasive, transdermal delivery device that is able to passively deliver L-DOPA directly and through the skin, to maintain a relatively constant drug level in the blood. Similarly, there is a need in the art for a skin-patch type non-invasive delivery device to administer other medications having similar profile characteristics (e.g., half-life, bioavailability, performance, etc.) to L-DOPA. Further, delivery of other clinically valuable macromolecular drugs remains challenging since their size restricts easy transport across the stratum corneum (SC), the dry, predominantly hydrophobic brick-and-mortar style cell arrangement of the topmost layer of the epidermis. The low permeability of the SC to important macromolecules and water-soluble drugs currently presents a major limitation of transdermal drug delivery systems that must be overcome to allow a broader spectrum of drug administration through the skin.

The unmet need for a simple-to-use, patient-applied, and patient-accepted skin-patch can be generalized to a more comprehensive means of dosing a broad range of unmodified drugs that reach the circulatory system in a completely non-invasive and non-intrusive manner. Such a transdermal approach alleviates first-pass hepatic effects and unpredictable gastrointestinal absorption, thereby allowing a reduction in the drug load being administered.

### SUMMARY

The embodiments herein describe a flexible transdermal skin patch that forms a technology platform that allows broad spectrum drug delivery and is not limited to L-DOPA alone. However, proof-of-concept will be established using L-DOPA delivery for Parkinson's Disease (PD) as the initial model drug, since it is the “gold standard” oral therapeutic for dopamine replacement in PD. The skin-patch technology platform has a broader significance that will allow safe, easy, painless, and effective delivery of medications, not requiring complex drug modifications or invasive devices, for a diverse population ranging from pediatric to geriatric. The technology relies on modifying the SC and not the drug itself by creating temporary micron-sized conduits, or micropores, that start at the skin surface and traverse through the SC and into the uppermost portion of the viable epidermis, thereby bypassing the problematic material properties associated with the SC. This painless tunneling directly into the interstitium implies lipophilic and hydrophilic therapeutic agents of macromolecular size can be delivered transdermally to reach the circulatory system.

In a first non-limiting exemplary embodiment, a flexible drug delivery patch, includes: a first sealing layer for directly contacting skin of a user on a first side thereof; a first polymer layer formed of a first flexible material including multiple electrically addressable microheating units located on a first side of the first polymer layer, wherein a second side of the first sealing layer is bonded to the first side of the first polymer layer; a second polymer layer formed of a second flexible material; a third polymer layer formed of the first flexible material, wherein the second polymer layer is located between the first and third polymer layers and is bonded thereto; a second sealing layer formed on a skin-facing side of the third polymer layer; wherein the first sealing layer, first polymer layer, second polymer layer, third polymer layer and second sealing layer forms sealed reservoirs therein, the sealed reservoirs containing one or more drugs; and further wherein, when activated, each of the multiple electrically addressable microheating units causes multiple microheating elements to open a micropore in a stratum corneum layer of the skin of the user, and to rupture a seal of one or more sealed reservoirs, thereby releasing interstitial fluid from the micropore which travels up to release the one or more drugs, the one or more drugs passing back through the micropore and through the skin of the user.

In a second non-limiting exemplary embodiment, a flexible drug delivery patch for non-invasively delivering at least one macromolecular drug directly to the circulatory system of a user, includes: at least a first flexible layer having multiple sealed reservoirs formed therein, the sealed reservoirs containing the at least one macromolecular drug entrapped within a stimuli-responsive hydrogel; means for accessing one or more sealed reservoirs from a first side thereof to activate the stimuli-responsive hydrogel to release the entrapped at least one macromolecular drug therefrom; and means for non-invasively accessing the circulatory system of the user and the one or more sealed reservoirs from a second side thereof to deliver the at least one macromolecular drug to the circulatory system of the user.

In a third non-limiting embodiment, a flexible drug delivery patch, includes: a first sealing layer for directly contacting skin of a user on a first side thereof; a first polymer layer formed of a first flexible material including multiple addressable microheating units located on a first side of the first polymer layer for non-invasively accessing a circulatory system of the user and for accessing one or more sealed reservoirs from a second side thereof to deliver at least one macromolecular drug to the circulatory system of the user, wherein a second side of the hydrophobic layer is bonded to the first side of the first polymer layer; a second polymer layer formed of a second flexible material; a third polymer layer formed of the first flexible material, wherein the second polymer layer is located between the first and third polymer layers and is bonded thereto; a second sealing layer formed on a skin-facing side of the third polymer layer; wherein the first sealing layer, first polymer layer, second polymer layer, third polymer layer and second sealing layer form sealed reservoirs therein, the sealed reservoirs; wherein the sealed reservoirs contain the at least one macromolecular drug entrapped within a stimuli-responsive hydrogel; and further wherein the third polymer layer includes multiple addressable microheating units located on a side thereof which is bonded to the second polymer layer to activate the stimuli-responsive hydrogel to release the entrapped at least one macromolecular drug therefrom.

### BRIEF SUMMARY OF THE FIGURES

Example embodiments will become more fully understood from the detailed description given herein below and the accompanying drawings, wherein like elements are represented by like reference characters, which are given by way of illustration only.

FIG. 1 illustrates the three basic prior art transdermal drug delivery pathways: intercellular, intracellular, and transappendageal;

FIG. 2 illustrates a temporary micropore generated by the patch for transdermal drug delivery in accordance with an embodiment herein (not to scale, but dimensions are approximately 40 microns diameter and 40-50 microns in depth);

FIG. 3a, 3b, 3c, 3d illustrate portions of a patch constructed in accordance with one or more embodiments herein;

FIGS. 4a, 4b, 4c illustrate exemplary microheating elements with PCL layer melting responses to varying voltages;

FIGS. 5a, 5b, 5c, 5d illustrate pore depth variance in accordance with voltage of microheater and time of application;

FIGS. 6a, 6b, 6c, 6d are snap shots of patch layer fabrication steps in accordance with embodiments herein;

FIGS. 7a and 7b illustrate nanostructured polymer and drug combinations in the form of nanofibers (FIG. 7a) and nanoparticles (FIG. 7b) for use in the reservoirs of the patch constructed in accordance with an embodiment herein;

FIG. 8 illustrates a doctor blade method for filling the reservoirs of the patch with a gel polymer and drug mixture constructed in accordance with an embodiment herein; and

FIG. 9 illustrates a final patch fabrication in accordance with one or more embodiments herein.

### DETAILED DESCRIPTION

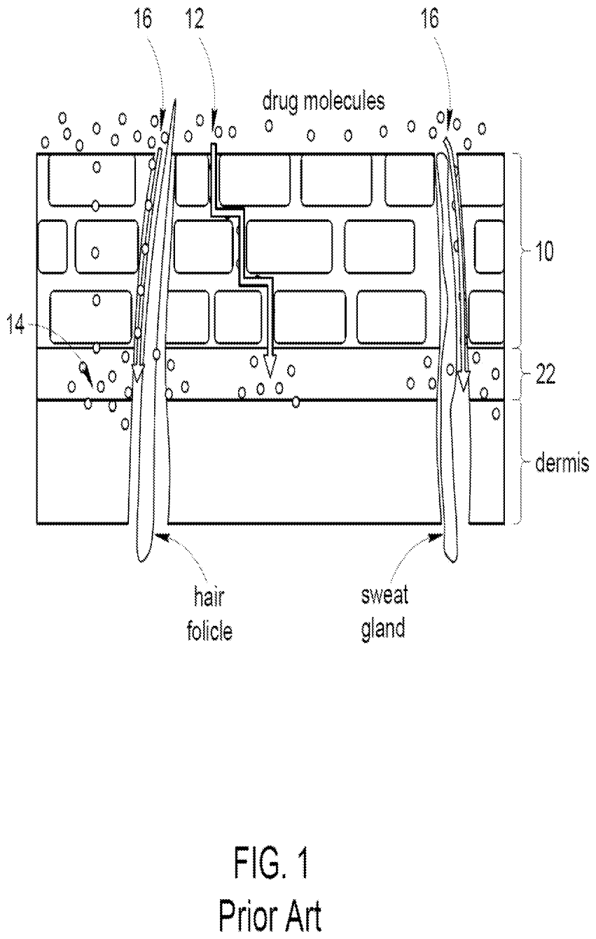

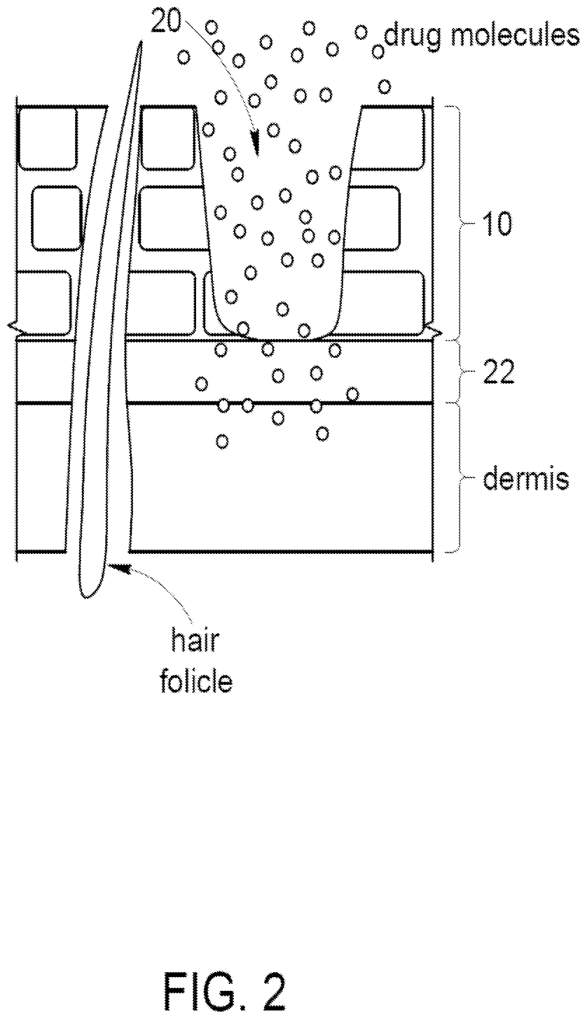

By way of further background, FIG. 1 is a prior art illustration of the three basic transdermal drug delivery pathways through the stratum corneum (“SC”) 10: intercellular 12, intracellular 14 and transappendageal 16. Each of these prior art pathways through the stratum corneum has significant limitations which limit one or more of the type and/or amount of the medications which may be passively delivered therethrough. Accordingly, referring to FIG. 2, the present embodiments utilize a process whereby the SC 10 is modified to effectively provide a micropore 20 directly to the interstitium and epidermis 22 and access to interstitial fluid (“ISF”).

The embodiments build on prior art processes and transdermal devices described in co-owned U.S. Pat. Nos. 6,887,202; 7,931,592; 8,568,315 and U.S. patent application Ser. No. 16/343,060 entitled Non-Invasive Passive Interstitial Fluid Collector, filed Oct. 20, 2017, Ser. No. 15/226,475 entitled Apparatus and Method For Delivery of Antimicrobial During a Transdermal Sampling and Delivery Process, filed Aug. 2, 2016, U.S. patent application Ser. No. 13/834,199 entitled Microfluidic Systems For Electrochemical Transdermal Analyte Sensing Using a Capillary-Located Electrode, filed Mar. 15, 2013, and issued as U.S. Pat. No. 10,004,434, each of which describe various transdermal devices intended to be used as or in connection with biosensors, wherein a micropore is formed in the SC using a microheater to access interstitial fluid and monitor biomolecules and biomarkers, e.g., glucose levels, therein. Details regarding the individual microheaters and arrays thereof were also described in additional co-owned patent filings including, but not limited to: U.S. patent application Ser. No. 13/459,392 entitled Electrochemical Transdermal Glucose Measurement System Including Microheaters and Process For Forming, filed Apr. 30, 2012. The patents and applications are incorporated herein by reference for all that they disclose.

The embodiments described herein include new features which transform the prior art biosensing patch to a passive drug delivery patch. And a particular application of the transformed passive transdermal drug delivery system or patch (“TDDS”) enables the delivery of L-DOPA (Levodopa) and Levocarb to maintain near-constant levels of medication to treat patients with Parkinson's disease.

The passive TDDS described in the preferred embodiments includes a compliant and flexible structure. The drug to be delivered will be stored within on-patch reservoirs as dissolvable solid micro and/or nanoparticles or micro and/or nanofibers, or as a hydrogel polymer matrix. Although technically possible, liquid drug storage is not preferred since evaporation would be a significant issue, as would ensuring a leak-proof patch when using flexible materials. The on-patch reservoirs are sealed with a compliant polymer to prevent ambient moisture from reaching the encapsulated drug, and the seals are easily opened during the drug delivery phase.

As described generally in the co-owned prior art, ISF is accessed by using a multiplicity of integrated micro-heating resistive elements (“microheater(s)”), fabricated as an array on the patch that sits on the surface of the skin. In the prior art biosensing devices, activation of a single microheater causes ISF to exude out from the thermally-generated micropore to wet a corresponding single biosensing site. Energizing a microheater directs a localized and highly controlled thermal pulse that ablates only the SC without affecting underlying viable cells, nerve endings, or capillaries. The temporary micropore that results provides direct access into the interstitium, yet causes no skin damage, no pain, and no sensation. For the present passive drug delivery patch embodiments, the microheaters will be relied upon to create micropores in the SC, but at the same time, to open the seals of the on-patch drug reservoirs. The ISF will passively rise to the skin surface due to the body's hydrostatic capillary pressure (i.e. without any form of active suction or vacuum extraction, or without resorting to troublesome iontophoresis). In accordance with careful design, the patch will be engineered using a combination of hydrophobic and hydrophilic polymers to direct the emergent ISF into the open drug reservoirs, mixing and dissolving the biocompatible medication-containing polymer for its release at the appropriate dosage.

As discussed further herein, the preferred embodiment is a point-of-care TDDS for dosing a broad range of unmodified drugs, including L-DOPA (Levodopa) and Levocarb (hereafter referred to generally as L-DOPA), that reach the circulatory system through micro-conduits created by the passive TDDS. In addition to Parkinson's disease drugs, the broader significance of the embodied TDDS is its applicability to a wider spectrum of water-soluble and macromolecular drugs, allowing for safe, easy, and effective transdermal delivery of medications for a variety of other treatable conditions.

The TDDS of the preferred embodiment enables hydrophilic L-DOPA to be delivered transdermally from the skin surface directly to the viable epidermis through temporarily generated micropores. Integrated microheaters on a wearable, non-invasive, fully-electronic TDDS, termed T-RxPatch (‘T-Rex’ patch), facilitate the creation of SC micro-conduits or micropores with no adverse inflammatory or pain response (as demonstrated by an early phase clinical trial). The T-RxPatch as described herein is a flexible polymeric TDDS with an array of microheaters, each producing ISF flow, which can mix with unmodified L-DOPA (or other drugs), stored in on-patch reservoirs, for reduced-dose, diffusive transdermal delivery through the micropores.

In the prior art biosensing devices, access to the ISF facilitated the sensing of biomolecules and biomarkers which exist in ISF, including those carried systemically by the blood and that are able to diffuse out from the capillaries. The sieve-like structure of the capillaries keeps the larger blood-borne constituents, such as platelets, red blood cells and white blood cells, from diffusing into the ISF provided that such large biomolecules are above the natural 60,000 Dalton (Da) capillary cutoff. Just as the ISF carries biomolecules and biomarkers that are able to pass through the sieve-like capillaries to the sensing device, the ISF can also be utilized as the medium for transport of the medication to the capillaries for its absorption into the circulatory system. ISF, the physiological liquid surrounding living cells of human tissues, is an extracellular fluid that is the main conduit for supplying nutrients to cells and removing any resulting metabolic waste. The T-RxPatch is able to access the interstitium and the ISF exudes to the skin surface in a completely passive manner, that is, without any form of active suction or vacuum extraction, and without resorting to problematic iontophoresis.

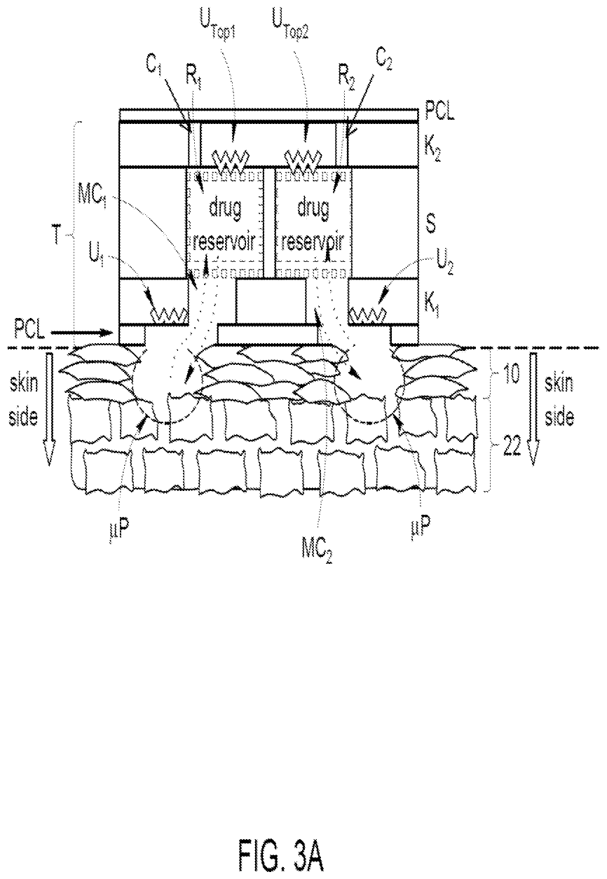

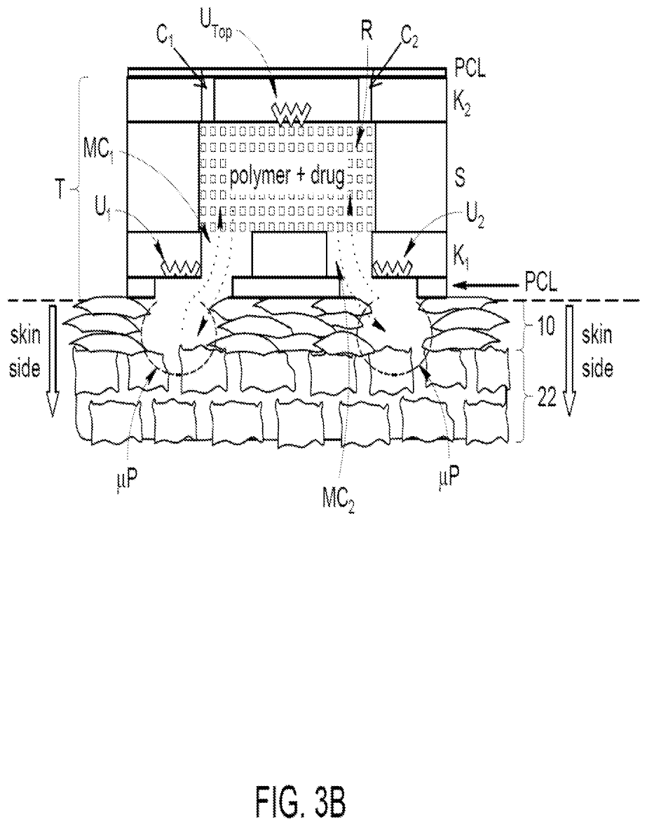

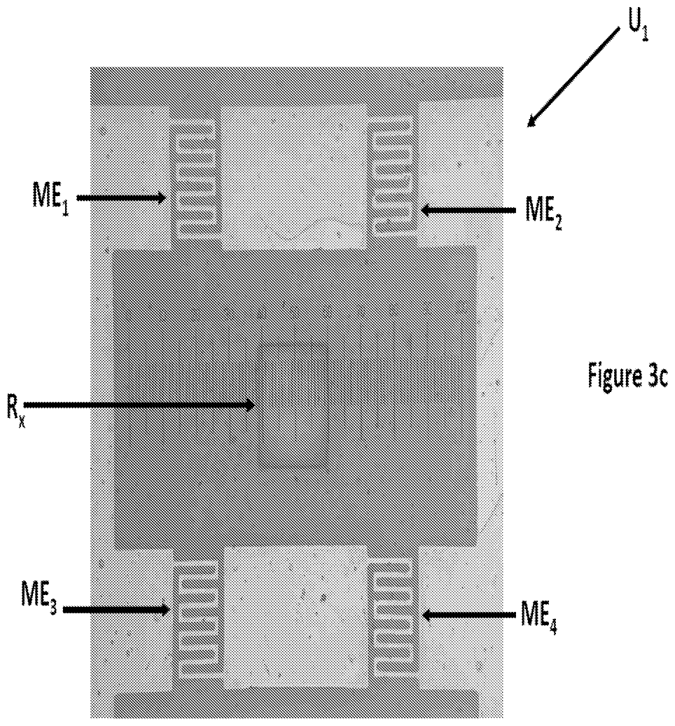

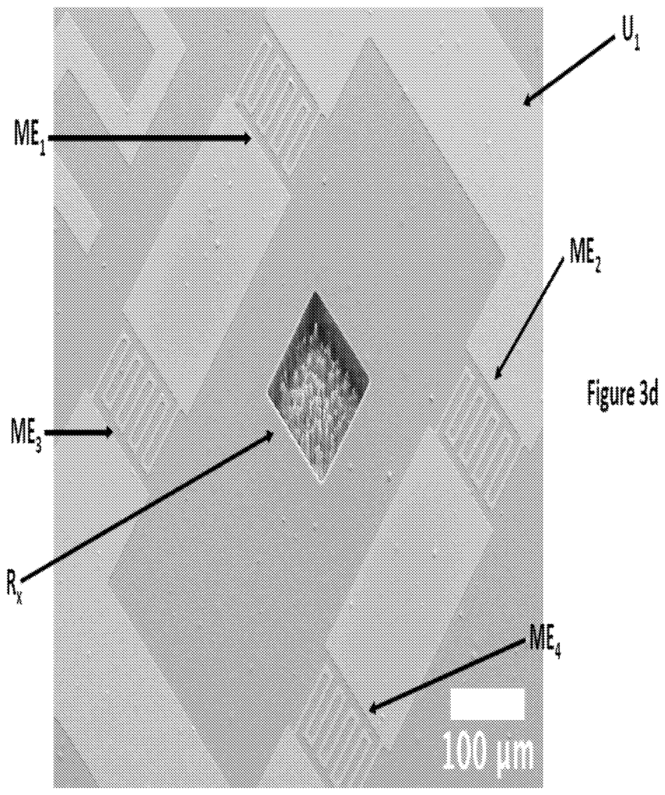

FIGS. 3a, 3b, 3c, 3d provide exemplary schematic and SEM views of a portion of a T-RxPatch constructed in accordance with the preferred embodiment. Initially, as shown in FIGS. 3a and 3b, the patch includes 5 layers of flexible materials having a combined thickness T of 5 mm or less (depending on height of reservoirs described later) and is situated on top of the surface of the skin. The bulk of the device is comprised of two polyimide (Kapton, by DuPont) layers K₁ and K₂ that sandwich a single layer of silicone S (polydimethylsiloxane, PDMS. The former finds use in flexible electronics (or flex circuits) since circuitry can be photolithographically patterned on its surface using gold metallic thin-films that are resilient even after bending (e.g. in folding laptop computers and the new foldable phones). PDMS is a soft, moldable polymer resin, or rubber, used in applications ranging from microfluidics to contact lenses, because of its biocompatibility.

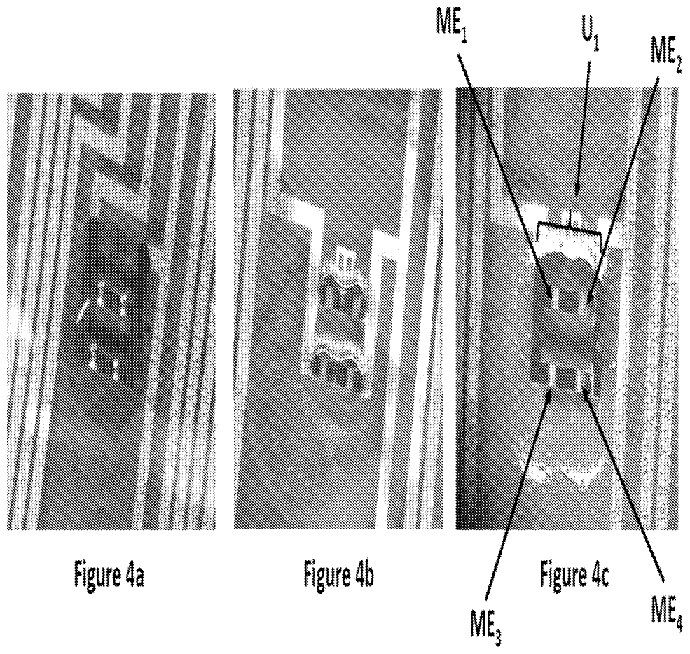

Further to FIG. 3a, microheating units U₁ and U₂ (of total patch units U₁₋ₓ) are integrated on the skin-side of a first Kapton layer, K₁, and energized to produce multiple micropores μP in only the SC. Kapton is a good thermal insulator meaning the heat dissipated by the microheating units U₁ and U₂ will be preferentially directed into the skin rather than upward into the patch. The final two layers are polycaprolactone (PCL) bonded to the top and bottom Kapton layers using click chemistry or, other adhesion means such as a biocompatible UV curable optical adhesive. These PCL layers effectively seal the drug reservoirs R₁₋ₓ. The skin-facing PCL layer is hydrophobic. And, prior to use, both PCL layers are continuous films across the Kapton layers blocking the preexisting openings to the drug reservoir in K₁ and the chimney openings C₁ and C₂ in K₂. However, since PCL effectively melts by retracting upon itself at a temperature of 60° C., activation of U₁ and/or U₂ to ablate the SC (typically, well above 60° C.) will also cause localized melting of the PCL (see FIGS. 4a, 4b, 4c), thereby opening access to/from pre-existing microchannels MC₁₋ₓ leading to/from the reservoir R₁₋ₓ to allow entry of the ISF and diffusion of the drug back through the micropores UP in the SC 10 and to the epidermis 22. In FIG. 3a, the PCL is shown in its melted state (see also FIG. 4c), after U₁ and U₂ have been activated, allowing the ISF exuding from the micropores to enter the patch reservoirs, R₁ and R₂, respectively, for mixing and dissolving the drug-containing polymer to release the drug. In a preferred embodiment, the dimensions of R₁ and R₂ are 2 mm×2 mm×300 μm (l×w×h).

Depending on the amount of ISF that results from each U, a single reservoir R may have access to ISF from multiple microchannels MC. By way of example only, in FIG. 3b, a ratio of MC to R of 2:1 can be achieved if the reservoir is designed by combining R₁ and R₂ from FIG. 3a together. For this case, the dimensions of R (designed as R₁+R₂ from FIG. 3a) spanning two MC is approximately 2 mm×4 mm. Alternatively, a ratio of MC to R of 4:1 is also contemplated, wherein the dimensions of R spanning four MC is approximately 4 mm×4 mm. The height of the R is also changeable depending on the drug dosage and the amount of ISF that is accessed. Estimates for the height of R, which is the height of the silicon layer, S, are approximately 0.5 mm to 2 mm.

In a preferred embodiment, a single T-RxPatch includes sixteen U (U₁₋₁₆). As shown in FIGS. 3c and 3d, each microheating unit U₁₋ₓ includes four individual microheating elements ME₁₋₄ and each individual element creates a micropore μP₁₋₄ (shown and labelled collectively as μP in FIG. 3a). By way of example only, each microheating elements ME₁₋₄ may be in the form of a serpentine trace which occupies an area of approximately 70 μm×100 μm with trace width being approximately 7 μm. Ideally, the access point between each MC₁₋₁₆, which leads to an R₁₋₁₆, and each U₁₋₁₆, is in the center of the U₁₋₁₆'s microheating elements ME₁₋₄ as shown in FIGS. 3b, 3c. The reservoirs R₁₋₁₆ shown in FIGS. 3b, 3c are not to scale for the purposes of a working patch. Accordingly, the ratio of ME to μP is 1:1 and the ratio of ME to U to MC is thus 4:1:1 in a preferred embodiment. Since the skin-facing PCL is a hydrophobic polymer, the ISF will be preferentially directed towards the pre-existing microchannels MC₁₋₁₆ in the Kapton layer K₁ and eventually upward into the PDMS-based reservoir R₁₋₁₆.

In FIGS. 4a, 4b and 4c, a single microheating unit U₁ with ME₁₋₄ is shown with a progression of voltages applied to the ME₁₋₄ to determine a preferred voltage that will evenly melt an 8-micron thick PCL covering the ME₁₋₄. The physical components are only labelled in FIG. 4c as this provides the clearest view. In FIG. 4a, 2-volts bulges PCL, In FIG. 4b, 4-volts unevenly melts PCL and in FIG. 4c, 5-volts evenly melts the PCL layer.

In its melted state, the ISF exuding from a micropore is directed up through a pre-existing microchannel and enters the patch reservoir for mixing and dissolving the drug-containing polymer or hydrogel for its release. Similarly, in a reverse, diffusive process, the unmodified drug, e.g., Levodopa/Levocarb, moves back through the microchannel and into the body through the thermally-generated micropores. One skilled in the art will appreciate that the number of micropores required to generate enough ISF to cause a release of the drug may vary.

In addition to the U_{1-X}, a separate microheating unit (or units), Uₜₒₚ₁₋ₓ, may be activated to melt both a localized portion of the upper PCL layer and the drug encapsulated polymer/hydrogel housed within the reservoir in the PDMS layer S to release the drug in certain configurations. The same microheating units may also activate thermal hydrogels to produce additional fluid which mixes with the released interstitial fluid to promote drug release back to user.

With respect to creation of each micropore, this relies on the associated integrated microheating element ME supplying a sufficient amount of thermal energy to effectively ablate only the SC without any thermal injury to the underlying tissue in order to avoid an adverse immunologic reaction. Nerve endings typically come up to the underside of the epidermis, which is estimated to be about 100 microns (μm) from the SC surface. Although subjective, accepted values of temperature for pain threshold at a nerve ending averages about 45 degrees C., while a 70 degree C. threshold exists for epidermal tissue damage, assuming the heating duration is at least several seconds long. Therefore, the pain-free aspect of the patch will be maintained if the heat pulse depth into the epidermis is held below 45 degrees C. Prior experiments have indicated that a 50-ohm micro-heating element and an application of a 3-volt pulse for only 30 msec will produce an ablation temperature of about 130° C. at the surface of the SC, while the temperature effectively decreases exponentially to safe levels within the viable epidermis. The resulting micropore that is generated in the SC is about 50 μm in diameter, and approximately 40 to 50 μm in depth.

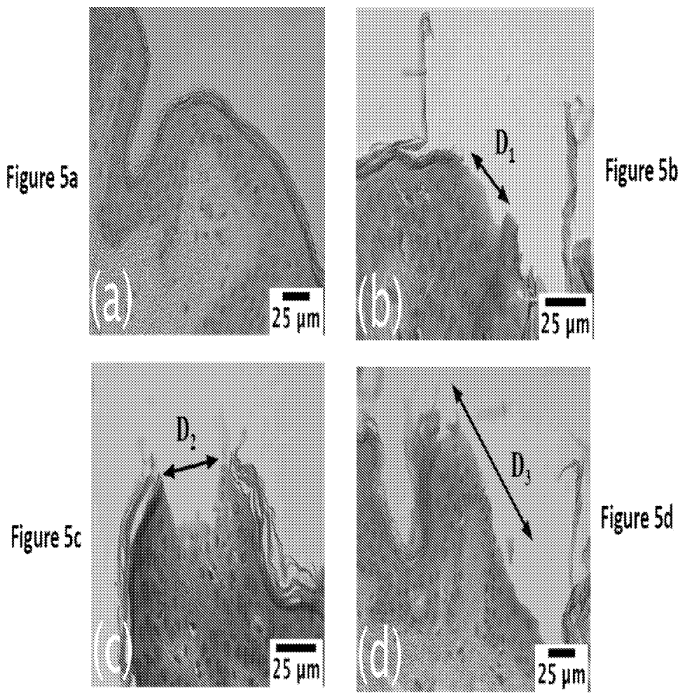

FIGS. 5a to 5d illustrate ablation depths in ex vivo human skin samples with hematoxylin and Eosin (H&E) staining to assess depth at 4V across a microheater for predetermined amount of time. Depths (D) were measured at 0 ms (FIG. 5a), 34 ms (FIG. 5b), 150 ms (Figured 5c) and 1000 ms (FIG. 5d). The photographs start to show measurable ablation depth D₁ of approximately 10 μm at 34 ms, followed by D₂ of approximately 25 μm at 150 ms and D₃ of 50 μm at 1000 ms.

Microheating units U can use a battery to apply a current to each ME₁₋₄. For initial animal tests, a wired signal will be used. In an alternative embodiment, a wireless patch allows a user to actuate the signal wirelessly or activation can be automated through a microcontroller. In a preferred embodiment, a flexible battery provides the necessary voltage. The power requirements are so low for the microheaters that most off-the-shelf flexible batteries can supply the power for a wearable device. The final device will support wireless data uploads to a smart device via short-range Bluetooth, WiFi, or similar near-field communication protocol.

The size of the drug reservoir will be fabricated to house sufficient amount of drug for administration to, at first, animal models, then for humans. With that in mind, Levodopa administration to a 25 g mouse would start at 10 mg/kg and would go up to 200 mg/kg, meaning 0.25 mg to 5 mg of Levodopa would be needed for the procedure. With the density of the PD drug being about 1500 mg/ml, the volume of Levodopa would range from 0.042 μl to 0.83 μl. To accommodate the medication, the reservoir size (l×w×h) would be about 2 mm×2 mm×300 μm. The area covered by the 2 mm×2 mm area encompasses the four microheater locations of a single Unit. Once optimal drug dosage for a mouse model has been ascertained, based on the same efficacy as that produced by a standard oral dose, dosing for humans can be determined.

As discussed above with respect to the preferred embodiment shown in FIG. 3a, the reservoirs R₁ and R₂ are in the PDMS layer S. But despite its many advantages, PDMS is inherently a hydrophobic material, and for the T-RxPatch, the PDMS-based reservoir should be hydrophilic. To this end, a modification to the PDMS layer as described in D. O'Brien et al., “Systematic Characterization of Hydrophilized Polydimethylsiloxane,” J. of Microelectromechanical Systems, 29(5), p. 1216, 2020, the teachings of which are disclosed here in their entirety, is used. In O'Brien, PDMS-b-PEO was identified as the optimal additive surfactant for long-term device stability in air storage due to its long-term stability and fast diffusivity in PDMS. The surfactant is simply mixed in with the PDMS prepolymer and curing agent and is cured in the usual manner (e.g., in an oven for a few hours, or overnight at room temp). This modified PDMS allows for pumpless fluid transport, and is quite simple to produce. One skilled in the art recognizes alternative methods for altering the nature of the PDMS layer S to make it hydrophilic.

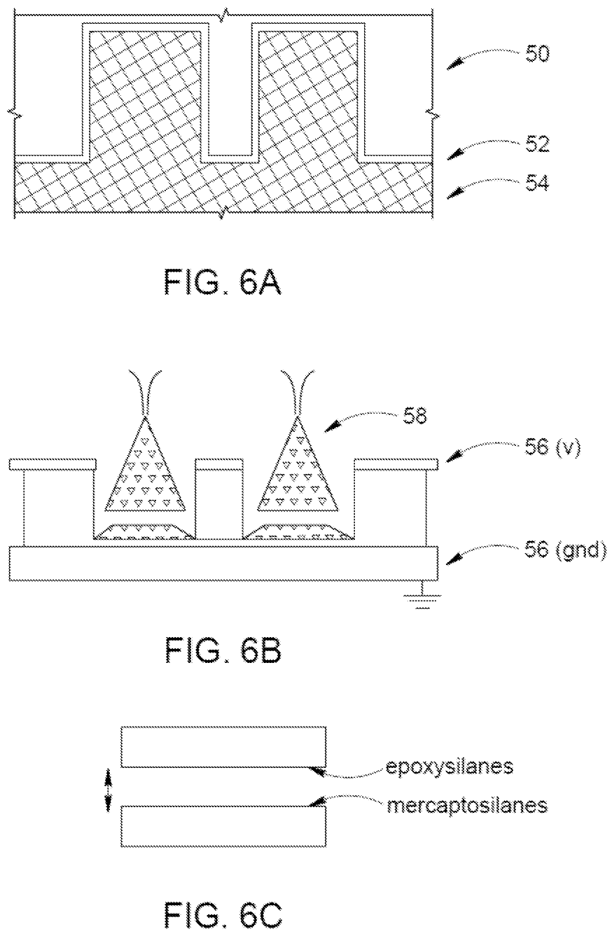

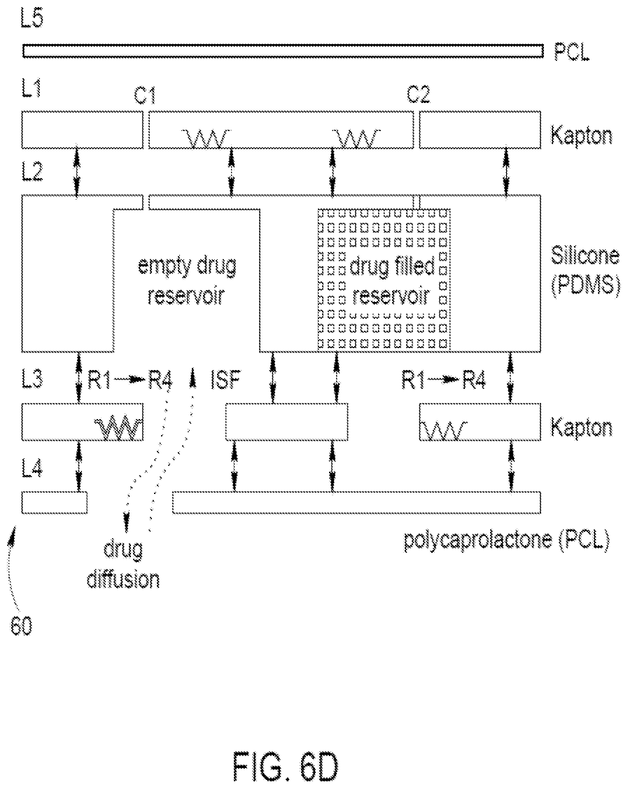

FIGS. 6a to 6c illustrate various phases of layer L1 to L5 development of final device FIG. 6d in accordance with at least one embodiment herein. Initially, for layers L1 and L3, the Kapton layer is attached to a Si handle wafer, gold microheaters and traces are deposited using a Ti or Cr adhesion layer with Ti sacrificial masking layer for DRIE hole etch. By way of example only, in an exemplary device, layers L1 and L3 are nominally 100 μm thick. For layer L2, in stage 1 shown in FIG. 6a, PDMS 50 is first engineered to be long-lasting hydrophilic (Ø) and molded on patterned SU-8 photopolymer 52 having a release layer (FDTS) 1H,1H,2H,2H perfluorodecyltrichlorosilane 54. As will be appreciated by one skilled in the art, the thickness of layer L2 can be varied, depending on the amount of drug needed. By way of example only, in an exemplary device, layer L2 is approximately 1 mm thick.

Next, in FIG. 6b, the structure of FIG. 6a is de-molded, inverted, voltage applied on removable metal mask 56 and the drug mixture 58 is added to the drug reservoirs using, e.g., electrospraying. Then L1, L2, L3 are bonded together using Kapton-PDMS irreversible bonding in vapor phase click chemistry as shown in FIG. 6c. Alternatively, a UV adhesive may be used to bond one or both of L1 and L3 to L2. The combination of layers L1, L2 and L3 forms the bulk of the drug reservoirs containing the drug mixture. Next, a continuous polycaprolactone film (which melts at 60° C.) is spin-coated onto the microheater side 60 of L3 to form L4. Finally, a second continuous polycaprolactone film (which melts at 60° C.) is spin-coated onto L1 to block air flow chimneys C₁ and C₂, seal the reservoirs and form layer L5. The materials selected for the fabrication of the flexible T-RxPatch must have thermal properties that can withstand the 130° C. heating requirement for approximately 30 ms. One skilled in the art will appreciate alternative materials which may be used in alternative embodiments.

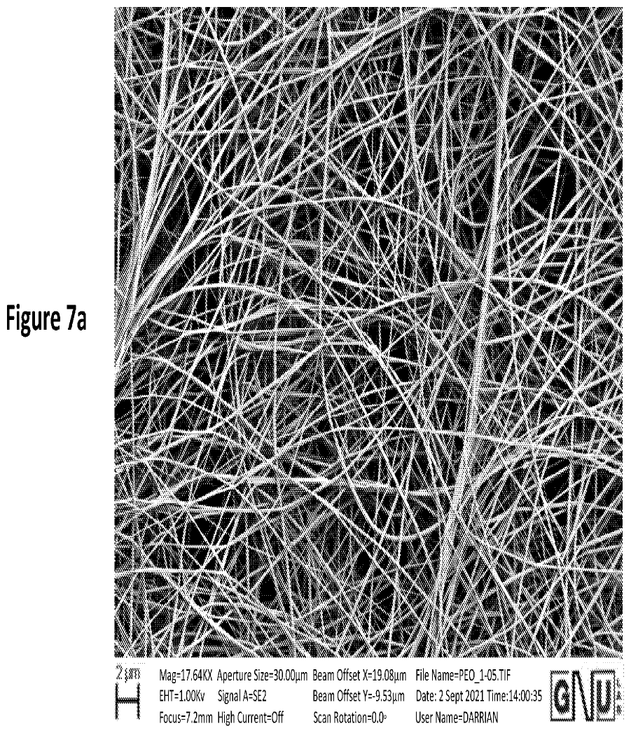

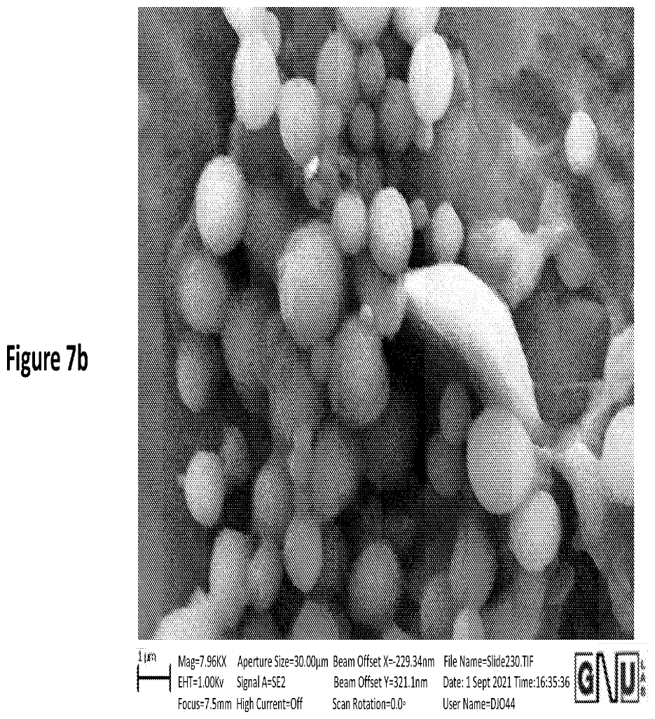

As referenced above, in the preferred embodiment, the drug is stored in a solid, brick-like or gel-form (e.g., hydrogel) within the PDMS reservoir. To that end, various techniques may be employed to prepare the drug for filling the reservoirs. In a first exemplary method, standard high-voltage electrospinning techniques for forming polyethylene oxide (PEO) nanofibers are mixed with the drug (see FIG. 7a) and in a second exemplary method, standard high-voltage electrospraying techniques for forming PEO nanoparticles are mixed with the drug (see FIGS. 6b and 7b). While in a third exemplary method, a process known as doctor blading, like stencil printing, is used to deposit a PCL polymer matrix containing the drug under test, directly into the reservoirs (FIG. 6). If the doctor blading/PCL method is chosen, a microheating unit Uₜₒₚ situated on the second Kapton layer K₂ layer (see FIG. 3a) will be used to assist in the release of the drug by melting the both the PDMS layer S and the entrapping polymer at 60° C.

Additionally, the hydrogel may be heat (or other stimuli-activated) activated by the microheating unit Uₜₒₚ to locally release additional fluid within the reservoir to facilitate release of the drug during use. Descriptions of exemplary heat activated hydrogels can be found in Huang et al., “Thermo-sensitive hydrogels for delivering biotherapeutic molecules: A review,” Saudi Pharmaceutical Journal 27 (2019) 990-999, which is incorporated herein by reference in its entirety. One skilled in the art will appreciate that the hydrogel may be activated by a different and/or additional environmental stimuli, e.g., pH, light.

With respect to the first and second exemplary filling methods, a high voltage (up to 30,000 volts) is applied between the needle tip, attached to a syringe containing the polymer/drug mix, and the grounded reservoir. A thin-film metal mask is placed on the Kapton layer with openings that correspond to the reservoir region, and connected to a positive potential to deflect the emerging nanofibers from the syringe tip into the reservoir. The same process can be used to deflect nanoparticles that emerge from the syringe tip under a higher voltage difference, resulting in a greater electric field. Essentially, the nanofibers that would normally emerge from the electrospinning setup experience a stronger electric field so that the fiber is essentially ripped up into smaller nanoparticles. The teachings in the following articles provide examples of processes which may be considered for electrospinning nanofibers or electrospraying nanoparticles and are incorporated herein by reference: A. Salim et al., Selective nanofiber deposition via electrodynamic focusing, 2008 Nanotechnology 19 375303; Lei, L., Gamboa, A. R., Kuznetsova, C. et al. Self-limiting electrospray deposition on polymer templates. Sci Rep 10, 17290 (2020); Suresh L. Shenoy, W. Douglas Bates, Harry L. Frisch, Gary E. Wnek, Role of chain entanglements on fiber formation during electrospinning of polymer solutions: good solvent, non-specific polymer-polymer interaction limit, Polymer, Volume 46, Issue 10, 2005, Pages 3372-3384; Morais, Alan Í. S. Morais et al. “Fabrication of Polymeric Microparticles by Electrospray: The Impact of Experimental Parameters.” Journal of functional biomaterials vol. 11(1), 4; 15 Jan. 2020; N. Bock et al, Electrospraying, a Reproducible Method for Production of Polymeric Microspheres for Biomedical Applications, Polymers 2011, 3, 131-149; and Daniel O'Brien, Makarand Paranjape, Biomedical Polymer Scaffolds Formed by Electrospinning and STRAND Technique, Bulletin of the American Physical Society, 2016.

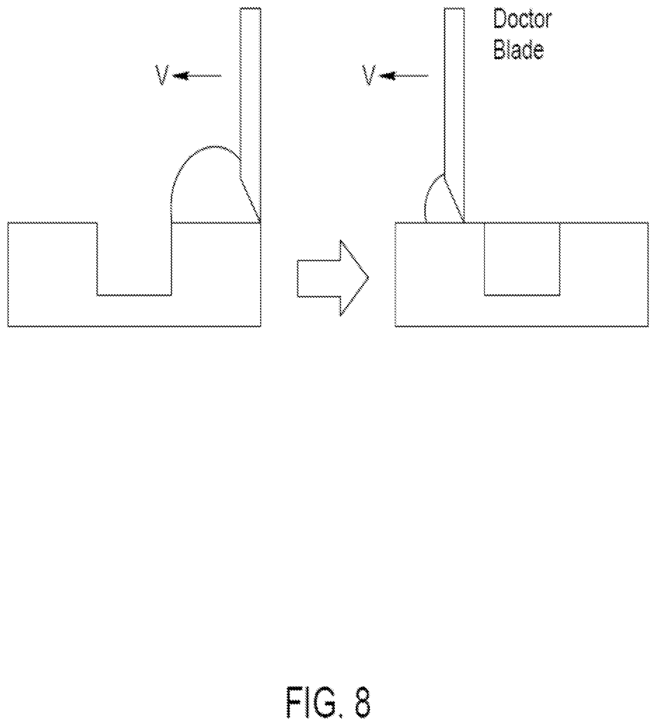

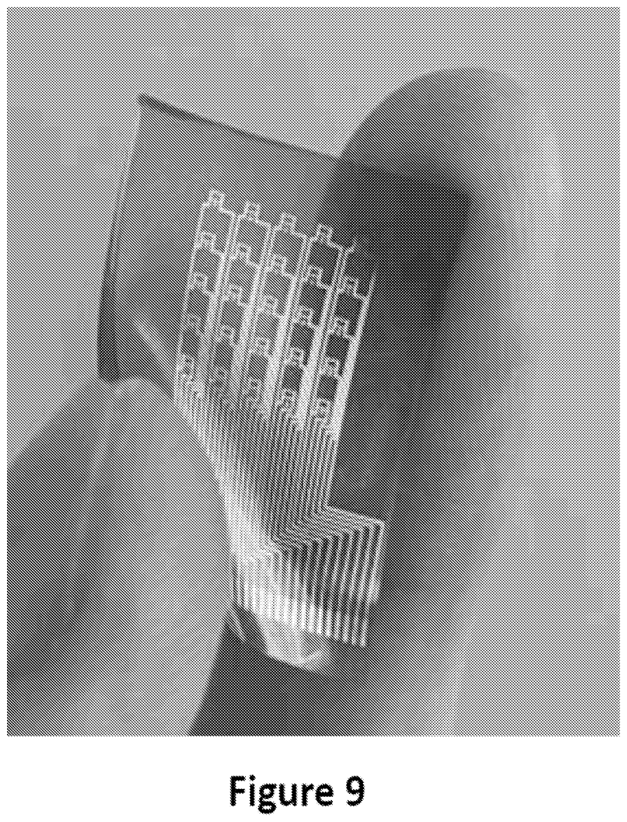

FIG. 8 is a photograph of an exemplary T-RxPatch prepared in accordance with the embodiments herein.

It will be appreciated by those skilled in the art that an adhesive layer may be included as part of the patch to secure the patch to the skin of the user.

In an alternative embodiment, the drug may be included in the reservoirs as a solid (brick-like) polymer matrix which is more slowly eroded by ISF for longer release rates, as compared to the more immediate release described with respect to the previous embodiments. In such an alternative embodiment, the same reservoir may be exposed to ISF multiple times, wherein different microheating unit U₁₋ₓ are activated at different times, e.g., serially, to erode the solid gel containing the drug over a predetermined amount of time.

The painless and non-intrusive nature of the flexible T-RxPatch to deliver drugs directly into the circulatory system, bypassing the gastrointestinal tract and liver, permits ease of use and improved quality of life. The T-RxPatch utilizes established micro-scale and nano-scale fabrication methods and exploits the advantages afforded by advanced batch processing methods inherent with integrated circuit fabrication. The T-RxPatch is completely reliant on simple resistive circuitry to generate the micropores and does not comprise any movable or mechanical structures, such as MEMS (microelectromechanical system) elements or micro-/nano-needles, to penetrate the skin surface. The T-RxPatch is a self-administered therapeutic device that is as simple as applying a Band-Aid. Although specific drugs are referenced herein, one skilled in the art will appreciate the broader applicability of the present embodiments to passive administration of numerous drugs which will significantly impact the treatment of patients and improve quality of life.

1. A flexible drug delivery patch, comprising:
a first sealing layer for directly contacting skin of a user on a first side thereof;
a first polymer layer formed of a first flexible material including multiple electrically addressable microheating units located on a first side of the first polymer layer, wherein a second side of the first sealing layer is bonded to the first side of the first polymer layer;
a second polymer layer formed of a second flexible material;
a third polymer layer formed of the first flexible material, wherein the second polymer layer is located between the first and third polymer layers and is bonded thereto;
a second sealing layer formed on a skin-facing side of the third polymer layer;
wherein the first sealing layer, first polymer layer, second polymer layer, third polymer layer and second sealing layer forms sealed reservoirs therein, the sealed reservoirs containing one or more drugs; and
further wherein, when activated, each of the multiple electrically addressable microheating units causes multiple microheating elements to open a micropore in a stratum corneum layer of the skin of the user, and to rupture a seal of one or more sealed reservoirs, thereby releasing interstitial fluid from the micropore which travels up to release the one or more drugs, the one or more drugs passing back through the micropore and through the skin of the user.

2. The flexible drug delivery patch according to claim 1, wherein the first and second sealing layers are polycaprolactone (PCL).

3. The flexible drug delivery patch according to claim 1, wherein the first polymer layer and the second polymer layer are comprised of a polyimide.

4. The flexible drug delivery patch according to claim 1, wherein the second polymer layer is comprised of a silicone rubber.

5. The flexible drug delivery patch according to claim 4, wherein the silicone rubber is engineered to be hydrophilic.

6. The flexible drug delivery patch according to claim 1, wherein the one or more drugs are entrapped within a dissolvable polymer matrix using one of nanoparticles or nanofibers and further wherein, the interstitial fluid from the micropore releases the one or more drugs by dissolution of the polymer matrix.

7. The flexible drug delivery patch according to claim 1, wherein the one or more drugs are entrapped within a stimuli-responsive hydrogel and further wherein, a combination of interstitial fluid from the micropore and stimuli-induced liquid from the stimuli-responsive hydrogel releases the one or more drugs from the stimuli-responsive hydrogel.

8. The flexible drug delivery patch according to claim 7, wherein the third polymer layer includes multiple addressable microheating units located on a side thereof which is bonded to the second polymer layer and further wherein the multiple addressable microheating units on the third polymer layer are activated to generate the stimuli-induced liquid from the hydrogel.

9. The flexible drug delivery patch according to claim 1, wherein the one or more drugs is a macromolecular drug.

10. The flexible drug delivery patch according to claim 9, wherein the macromolecular drug is L-3,4-dihydroxyphenylalanine (L-DOPA).

11.-20. (canceled)
